(19) United States
(12) Patent Application Publication
(10) Pub. No.: US 2025/0249219 A1
(43) Pub. Date: Aug. 7, 2025

(54) MEDICAL DEVICE FOR GUIDEWIRE PLACEMENT AND RELATED METHOD

(71) Applicant: The Regents of the University of Michigan, Ann Arbor, MI (US)

(72) Inventors: Mostafa Abdel-Hamid, Ann Arbor, MI (US); Matthew F. Grzywinski, Ann Arbor, MI (US); Maxwell Raithel, Ann Arbor, MI (US); Maddisen Robirds, Ann Arbor, MI (US); Elizabeth M. Dulzo, Novi, MI (US); Maxwell Lee, Ann Arbor, MI (US); Meredith Wojewuczki, Fort Wayne, IN (US)

(21) Appl. No.: 18/856,783

(22) PCT Filed: Apr. 13, 2023
(86) PCT No.: PCT/US2023/018481

§ 371 Date: Oct. 14, 2024

Related U.S. Application Data
(60) Provisional application No. 63/331,195, filed on Apr. 14, 2022.

(51) Int. Cl. A61M 25/09

(52) U.S. Cl. CPC A61M 25/09041; A61M 2025/09116; A61M 2205/3344; A61M 2205/3375; A61M 2210/101

(57) ABSTRACT

A medical device includes a needle coupled with a syringe and is configured to feed a guidewire through the needle and out of a tip of the needle with only one hand and without decoupling the needle from the syringe. A user can feed the guidewire through the device using the same hand holding the device and using the same hand that applies a vacuum in the syringe. The device frees up the user's other hand to control the position of an ultrasonic transducer to continuously visualize all steps of vascular access, including feeding of the guidewire into the blood vessel or other hollow cavity of a patient.

### TECHNICAL FIELD

The present disclosure relates generally to medical procedures involving accessing blood vessels and other fluid-filled areas of the body and to devices used in such procedures.

### BACKGROUND

Vascular access is a common medical procedure often required for time-sensitive administration of vasoactive agents or other life-sustaining therapies in emergency medicine and critical care environments. But placement of catheters, such as a central veinous catheter, can be difficult and further complicated by patient body habitus, limited mobility, abnormal vasculature, and busy practice environments. The Seldinger technique was developed in the 1950s and has remained the standard for obtaining central venous access. This technique has evolved little since its introduction and was developed before point-of-care ultrasound was available. Even when ultrasound-guided, the Seldinger technique has several limitations. For instance, the guidewire or catheter cannot be actively visualized when advanced into the blood vessel or other hollow organ because both of the medical provider's hands must be used to transition from syringe aspiration to guidewire insertion, requiring the provider to interrupt the ultrasound imaging, which is typically performed by the provider's non-dominant hand while the dominant hand is used for the aspiration. This transition also requires fine motor skills and has significant potential for error, such as blind passage of the guidewire outside the target vessel. Potential complications of such blind guidewire passage can include procedural failure, neurovascular injury, arteriovenous fistulas, or hematomas.

### SUMMARY

Embodiments of a medical device include a needle coupled with a syringe, the device being configured to feed a guidewire through the needle and out of a tip of the needle with only one hand and without decoupling the needle from the syringe.

The medical device may include a handle supporting the syringe and a guidewire feeder supported by the handle. The guidewire feeder includes a body, a guide channel extending through the body, and a wheel. The wheel is operable by a hand of a user to feed the guidewire along the guide channel and through the needle while the same hand is holding the device by the handle.

The medical device may include a handle supporting the syringe and a guidewire feeder supported by the handle. The guidewire feeder includes a body and a guide channel extending through the body. The guide channel opens on an exterior surface of the body such that a hand of a user can contact and move the guidewire in the channel to feed the guidewire along the guide channel and through the needle while the same hand is holding the device by the handle.

The medical device may include a handle supporting the syringe and an actuator supported by the handle. The actuator is operable by a hand of a user to decrease pressure in the syringe when the tip of the needle is blocked and while the same hand is holding the device by the handle. The medical device may further include a guidewire feeder supported by the handle. The guidewire feeder is operable by the same hand of the user to feed the guidewire along a guide channel and through the needle.

The medical device may include a connector having first and second branches coupled with the needle. The first branch is coupled with the syringe and the guidewire is fed to the needle via the second branch. The device may further include a check valve along the second branch to prevent external fluid flow into the connector via the second branch when pressure is decreased in the connector.

The medical device may include a handle supporting the syringe, a guidewire feeder operable to feed the guidewire through the needle, an actuator operable to decrease pressure in the syringe when the tip of the needle is blocked, a connector having first, second, and third ends interconnected by an internal volume of the connector, and a check valve. The first end of the connector is coupled with the syringe, the second end of the connector receives the guidewire, the third end of the connector is coupled with the needle, and the check valve prevents external fluid flow into the internal volume through the second end of the connector when pressure is decreased in the internal volume of the connector. The feeder may include a wheel operable by a hand of a user to feed the guidewire along a guide channel of the device, into the second end of the connector, through the check valve, out of the third end of the connector, and through the needle while the same hand is holding the device by the handle. The actuator may include a trigger operable by a hand of a user to apply force to a plunger of the syringe to aspirate a fluid-filled part of the body when the tip of the needle accesses the fluid-filled part of the body through a wall of the fluid-filled part of the body while the same hand is holding the device by the handle.

The medical device may include a pressure transducer operable to detect pressure changes in the syringe during use of the device.

Embodiments of a method include the following steps, each of which are performed using only one hand:
(a) guiding a tip of a needle through a wall of a fluid-filled part of a body of a person;
(b) maintaining a partial vacuum in the needle before and during step (a);
(c) feeding a guidewire into the fluid-filled part through the needle; and
(d) maintaining a position of the tip of the needle in the fluid-filled part during step (c).

The method may include use of real-time imagery during step (a). The imagery is obtained at a location controlled by a different hand.

The method may include use of real-time imagery during steps (a) and (c). The imagery is obtained at a location controlled by a different hand, and the imagery is continuous between steps (a) and (c). The real-time imagery is obtained via an ultrasound transducer held in the different hand.

The needle may be coupled with a syringe during step (a), and step (c) may be performed without decoupling the needle from the syringe.

Step (b) may be performed using a digit of the one hand, and step (c) may be performed using a different digit of the one hand.

The fluid-filled part of the body may be a blood vessel or a pleural cavity.

Embodiments of a method of obtaining vascular access include the following steps:
(a) guiding a tip of a needle into a blood vessel of a person; and
(b) feeding a guidewire into the fluid-filled part through the needle,
wherein real-time imagery is continuously provided to a person performing the method during and between steps (a) and (b).

Steps (a) and (b) may be performed using only one hand, and the imagery may be provided by an ultrasound transducer held in a different hand.

The method may include the following steps:
maintaining a partial vacuum in the needle before and during step (a); and
maintaining a position of the tip of the needle in the blood vessel during step (b),
wherein steps (a) and (b) and each maintaining step are performed using only one hand.

Steps (a) and (b) may be performed while the needle is coupled with a syringe.

### BRIEF DESCRIPTION OF THE DRAWINGS

FIG. 1 is a cutaway and partial cross-sectional side view of an illustrative medical device;

FIG. 2 is an exploded side view of the device of FIG. 1;

FIG. 3 is an isometric view of an actuator of the device of FIGS. 1 and 2;

FIG. 4 is a photographic image of a prototype of the device of FIGS. 1 and 2;

FIG. 5 is a cutaway and partial cross-sectional side view of a variation of the medical device of FIGS. 1 and 2;

FIG. 6 is a schematic depiction of the device prior to use in an ultrasound-guided vascular placement method;

FIG. 7 is a schematic depiction of the device after piercing the skin in the placement method;

FIG. 8 is a schematic depiction of the device after piercing the blood vessel in the placement method;

FIG. 9 is a schematic depiction of the device after feeding a guidewire into the blood vessel;

FIG. 10 is a schematic depiction of the device upon withdrawal from the blood vessel; and

FIG. 11 is a schematic depiction of a catheter being guided into position in the blood vessel along the placed guidewire.

### DESCRIPTION OF EMBODIMENTS

Described below is a medical device and methods of using the device that can provide real-time imaging during advancement of the guidewire and less complex manipulation of the aspirating syringe and needle. The device can be a single-use adjunct to existing central and arterial line kits and may combine the needle, guidewire, and syringe found in conventional procedure kits into a single assembly that remains an assembly throughout the procedure.

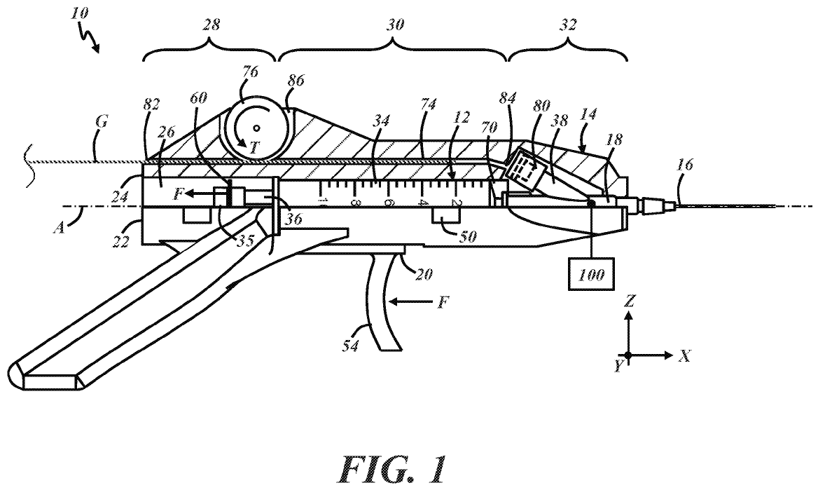

FIG. 1 is a cutaway and partial cross-sectional view of an illustrative medical device 10 useful for one-handed placement of a guidewire into a hollow portion of the body of a patient, such as a blood vessel, a pleural cavity, or any other fluid-filled part of the body where access must be obtained through a wall defining the hollow portion. The device includes a syringe 12 supported within a housing 14. A needle 16 is coupled with the syringe 12 via a connector 18. An actuator 20 is supported by the housing 14 for translational movement relative to the housing in the axial or longitudinal (X) direction.

The housing 14 includes a handle 22 and a feeder 24 that together define a cavity 26 in which the syringe 12 is supported. The cavity 26 has a rear portion 28, a middle portion 30, and a forward portion 32 arranged along a central axis A of the device 10. A barrel 34 of the syringe 12 is supported at a fixed location in the housing 14 at the middle portion 30 of the cavity 26, and the connector 18 is supported at a fixed location in the housing at the forward portion 32 of the cavity. The rear portion 28 of the cavity 26 provides a space in which a slotted portion 35 of the actuator 20 and a plunger 36 of the syringe 12 are free to move in axial translation. At least a portion of the rear and middle portions 28, 30 of the cavity 26 are cylindrical in this example, with the handle 22 and the feeder 24 each providing half of those portions of the cavity. At least a portion of the lower part of the forward portion 32 of the cavity 26 is semicylindrical where the connector 18 is supported. The upper part of the forward portion 32 of the cavity 26 is shaped to accommodate a branch 38 of the connector 18.

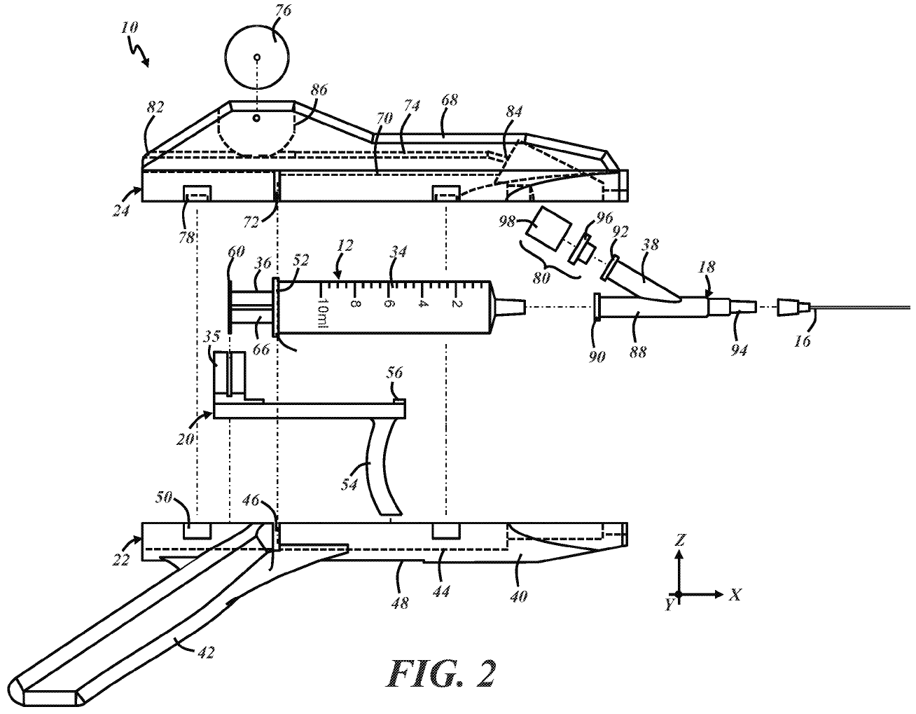

FIG. 2 is an exploded side view of the device 10 of FIG. 1. The handle 22 is the bottom piece of the housing 14 in this example and includes an elongated body 40 and a pistol-like grip 42 extending away from the body at an angle. The grip 42 is configured to lie along the palm of the hand of a user with one or more fingers wrapped at least partly around the grip. The body 40 of the handle 22 supports the other components of the device 10 when assembled and includes an axial recess 44 extending over the full length of the body 40, a transverse or radial slot 46, a central opening 48 along the bottom of the body and forward of the grip 42, and attachment features 50 for attaching the feeder 24 to the handle.

The axial recess 44 defines a portion of the cavity 26 of FIG. 1 and has a semicircular cross-section in a Y-Z plane complimentary to the shape of the barrel 34 of the syringe 12 and the connector 18 where each of those components is supported by the body 40. The recess 44 may also have a semicircular cross-section where the slotted portion 35 of the actuator 20 and the plunger 36 of the syringe 12 are located.

The transverse slot 46 is formed parallel with a Y-Z plane and defines an axial gap sized to fit a flange 52 at the plunger end of the barrel 34 of the syringe 12. The slot 46 functions to axially constrain the barrel 34 of the syringe 12 when the flange 52 is in the slot and the plunger 36 is moved relative to the barrel.

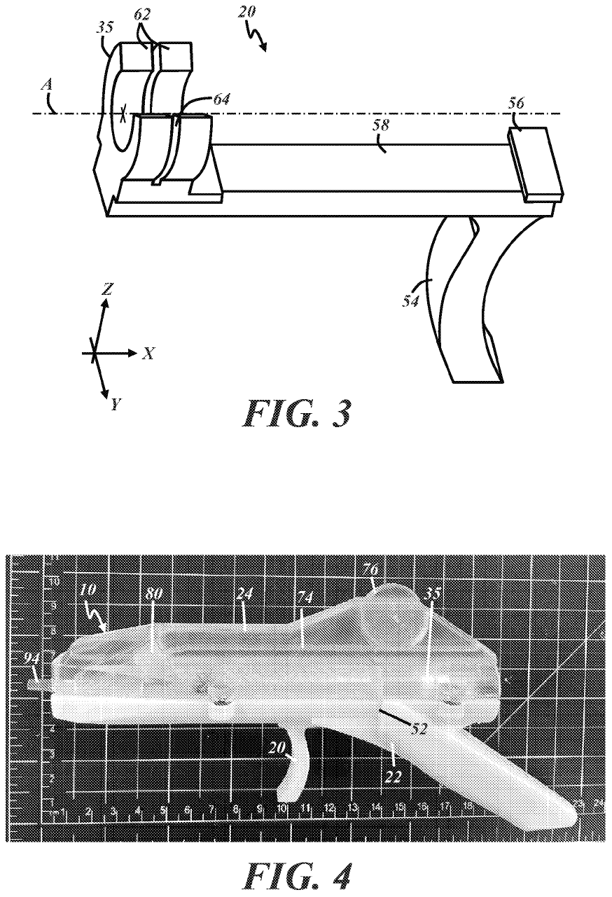

A trigger portion 54 of the actuator 20 protrudes through the central opening 48 along the bottom of the body 40 and is positioned so that a user's finger can pull the actuator 20 toward the grip 42 when the user's hand is holding the handle 22 by the grip. The actuator 20 is slidingly coupled with the handle 22 in this example by transversely extending tabs 56, as best illustrated in FIG. 3. As shown in FIG. 3, the actuator 20 includes an axially extending slide portion 58 from which the tabs 56 extend in a transverse (Y) direction. The slotted portion 35 and the trigger portion 54 each extend away from the slide portion 58 in opposite (Z) directions.

The tabs 56 are received by slots, grooves, or other guide features of the handle 22 (not shown) in this example. Side or bottom surfaces of the slotted portion 34 may also slide along mating surfaces of the handle 22. Alternatively, the handle 22 may include tabs or other protrusions configured to slide along slots or other recesses formed in the actuator 20. Other types of actuators are possible and are not necessarily limited to pure translational movement. The actuator 20 need only be capable of moving the plunger 36 of the syringe 12 relative to the barrel 34 of the syringe in response to user input. In this example, the user input is a force F applied to the trigger portion 54 of the actuator 20 such that the same resultant force F is applied to a tab 60 of the plunger 36, as shown in FIG. 1. The actuator 20 could include a button or touch-sensitive element that moves the plunger 36 through electromechanical means, for example, or could include levers, cams, gears, etc. designed to change the amount or direction of the user applied force when transmitted to the plunger 36.

In the illustrated embodiment, the slotted portion 35 of the actuator 20 is formed from two partial rings 62 spaced apart from each other in the axial (X) direction to form a slot 64. The axial dimension of the slot is sized to receive the disc-shaped tab 60 (see FIG. 2) of the plunger 36 of the syringe 12. Each partial ring has a C-shape in this example, with the open end of the C-shape facing upward. The internal radius of the C-shape is complimentary to and accommodates the radial dimensions of a shaft portion 66 (see FIG. 2) of the plunger 36.

With continued reference to FIGS. 1 and 2, the feeder 24 is the top piece of the housing 14 in this example and includes a main body 68, an axial recess 70 extending over the full length of the body 68, a transverse or radial slot 72, a feed channel 74, an actuator 76, and attachment features 78 for attaching the feeder 24 to the handle 22. In the illustrated example, the attachment features 50, 78 are bosses extending away from the respective handle 22 and feeder 24 in the transverse (Y) direction with recesses and/or apertures to accommodate reversible fasteners such as screws, bolts, magnets, etc. Other non-limiting examples of attachment features include integral snap tabs, adhesive joints, welds, clamps, etc.

The axial recess 70 is formed in the body 68 and defines a portion of the cavity 26 of FIG. 1. The recess 70 has semicircular cross-sections in a Y-Z plane complimentary to the shape of the barrel 34 of the syringe at the middle portion 30 of the cavity 26 and accommodating the slotted portion 35 of the actuator 20 and the plunger 36 of the syringe 12 at the rear portion 28 of the cavity 26. The portion of the axial recess 70 corresponding to the forward portion 30 of the cavity 26 is shaped to accommodate the branch 38 of the connector 18 and a valve assembly 80 attached to the connector 18.

The transverse slot 72 is substantially the same as the transverse slot 46 of the handle 22. The slot 72 is formed in the main body 68 parallel with a Y-Z plane and defines an axial gap sized to fit the flange 52 of the syringe 12. Together with the slot 46 of the handle 22, the slot 72 functions to axially constrain the barrel 34 of the syringe 12 when the flange 52 is in the slot and the plunger 36 is moved relative to the barrel.

The feed channel 74 is formed through the main body 68 and has a cross-section sized to accommodate a guidewire G (FIG. 1). When the guidewire is fed into the device 10 through a first open end 82 of the feed channel 74, the channel serves to guide the guidewire to an opposite second end 84 opening on the portion of the axial recess 70 and cavity 26 that houses the connector 18. In the illustrated example, a majority portion of the channel 74 is an axial channel, and a portion of the channel at the second end 84 is angled toward the central axis A.

The actuator 76 is a thumbwheel in this example and is rotatable about a transverse (Y) axis at a fixed position along the body 68. The actuator 76 is housed in a recess 86 formed along an exterior surface of the body 68 with only a portion of the actuator (about one quarter of the angular extent of the thumbwheel in this case) protruding beyond the exterior surface. The illustrated actuator 76 is positioned so that it makes physical contact with the guidewire G when the guidewire is in the feed channel 74 such that the guidewire is lightly pressed between the actuator 76 and an opposing wall 85 of the channel. The actuator 76 is also positioned so that the user's thumb can rotate the actuator when the user is holding the handle 22 via the grip 42. When the actuator 76 is rotated counterclockwise (as oriented in the figures) with the guidewire G in the channel 74, the guidewire is fed along the channel 74 toward the needle end of the syringe 12.

Where the actuator 76 is a thumbwheel, its perimeter may include friction enhancement, such as a toothed or grooved surface, a knurled surface, or an elastomeric surface. Other types of actuators are possible and are not necessarily limited to rotational movement. The actuator 76 need only be capable of moving the guidewire ( ) along the channel 74, through the connector 18, and through the needle 16 in response to user input. In this example, the user input is a torque T applied to the actuator 76 such that a resultant translation force proportional to the radius of the thumbwheel is applied to the guidewire G. The actuator 20 could include a button or touch-sensitive element that moves the guidewire via electromechanical means, for example, or could include levers, cams, gears, etc. designed to change the amount the user applied force transmitted to the guidewire.

The syringe 12 may be a commonly available medical grade syringe, such as a 10 ml syringe with a polypropylene barrel 32 and flange 52 and a polyisoprene seal on the end of the plunger housed in the barrel 32. While the illustrated construction of the device 10 is convenient in that a standard-sized syringe 12 can be used or be interchanged in the housing 14, it is contemplated that syringe functions may be integrally formed into other parts of the device. A portion of the cavity 26 of the device 10 may for example be employed in the manner of the barrel of a syringe and/or in place of the illustrated connector 18 to reduce the number of individual pieces required to make the device 10. The device 10 may for instance define an internal volume (as in the volume of a syringe barrel), where the internal volume is variable via movement of a piston (as in the plunger of a syringe).

The illustrated connector 18 is a Y-connector including a first branch 88 and a second branch 38 coupled with the needle 16. In this example, the first branch 88 is coupled with the syringe 12 (e.g., via a luer lock). The second branch 38 is positioned to receive the guidewire G from the feed channel 74 and route the guidewire to the hollow interior of the needle 16. Stated differently, the connector 18 has a first end 90 coupled with the syringe 12, a second end 92 that receives the guidewire from the feed channel 74, and a third end 94 coupled with the needle 16 (e.g., via a luer lock). The first, second, and third ends 90-94 of the connector 18 are interconnected by an internal volume of the connector.

The valve assembly 80 is located along the second branch 38 of the connector 18 and is attached at the second end 92 of the connector in this example. The valve assembly 80 includes a check valve 96 in a housing 98. The valve 96 operates to prevent external fluid (e.g., air) from being drawn into the connector 18 and syringe 12 when the internal volume of the syringe is at a reduced pressure. For example, during a method of guidewire placement in a blood vessel or other body cavity, the user of the device 10 may use the actuator 20 after the needle 16 pierces a patients skin but has not yet pierced a wall of the blood vessel to cause a reduced pressure (i.e., below atmospheric pressure) in the syringe 12, connector 18, and needle. The valve 96 serves to maintain that reduced pressure until blood vessel is pierced and aspirated by the reduced pressure.

The valve 96 is also configured to permit the guidewire G to be fed through the valve after aspiration, then through the connector 18 to the needle 16. In one embodiment, the valve 96 is a silicone valve that is sufficiently rigid to seal-off the end 92 of the connector 18 when the pressure of the internal volume of the connector is reduced and sufficiently flexible to permit the guidewire to push the valve open under the power of the actuator 76 of the feeder 24. In one embodiment, the valve 96 is supported and coupled to the connector 18 via a snap-on luer lock ring as the housing.

The device 10 may also include a pressure transducer 100, illustrated schematically in FIG. 1, arranged to detect fluid pressure and/or changes in fluid pressure in the internal volume of the connector 18, syringe 12, and needle. Such a transducer 100 can be placed in communication with a data logger or controller to record or indicate to the user in real-time when aspiration has been achieved without relying on visual cues.

Like the syringe, the needle 16 and connector 18 may be commonly available components. The illustrated connector 18 is a Y-connector with the first and second ends 90, 92 being luer receivers and the third end 94 being a slip end configured to be received by a mating component of the needle 16. In one embodiment, the needle 16 is an 18-gauge stainless steel needle having a length of 6.35 cm (2.5 inches). The guidewire G is also common in vascular access kits and may be a 60 cm stainless steel wire having a diameter of 0.81 mm (0.003 inches). The housing 14, including the handle 22 and the body 68 of the guidewire feeder 24, may each be molded as a single piece from a thermoplastic resin such as polypropylene. Each of the actuators 20, 76 may be molded separately from the housing 14 as individual pieces from a thermoplastic resin such as polypropylene as well.

A working prototype medical device 10 has been constructed using 3D-printed components and is illustrated in FIG. 4. In this example, the handle 22 and trigger actuator 20 were 3D-printed in white poly (lactic acid) resin (PLA), while the feeder 24 and thumbwheel actuator 76 were 3D-printed in translucent PLA. The third end 94 of the connector 18 is visible protruding from the front end of the housing. The valve assembly 80 is visible through the housing, as are the slotted portion 35 of the trigger actuator and the feed channel 74. The thumbwheel actuator 76 is mounted to the body of the feeder 24 on a stainless-steel axle. The flange 52 of the syringe is shown protruding through the housing via the transverse slots 46, 72 of the housing 14. The overall size of the prototype device 10 without the needle is just over 200 mm in length, 30 mm in width, and 95 mm in height. These dimensions are of course non-limiting and are presented here only to demonstrate actual reduction to practice of an embodiment of the disclosed device 10.

FIG. 5 is a cutaway and partial cross-sectional view of another embodiment of the medical device 10 useful for one-handed placement of a guidewire. This example includes the same syringe 12, needle 16, connector 18, and actuator 20 as in the previous example. The housing 14 also includes the same handle 22 and defines the same or similar cavity 26 for housing the syringe 12, but the feeder 24 is different.

As in the previous example, the feeder 24 of FIG. 5 includes a main body 68, an axial recess 70 defining part of the cavity 26, and a guidewire feed channel 74 extending axially through the main body 68 from a first or back end 82 to an opposite second end 84 toward the front of the device. But in this embodiment, the thumbwheel actuator 76 of FIGS. 1 and 2 is omitted, and movement of the guidewire G is performed directly by one of the user's digits, which in this case is the user's thumb, as shown in phantom in FIG. 5.

Here, a portion of the feed channel 74 is open along the upper outer surface of the feeder 24 to permit the user's thumb to contact the guidewire G at a middle section 65 of the feed channel located between a rearward section 75 a forward section 85 of the feed channel. The rearward section 75 of the feed channel 74 includes the first open end 82 of the feed channel, and the forward section 85 includes the second open end 84 at the connector 18. The middle section 65 of the feed channel 74 may be in the form of an upward-facing groove that helps guide the guidewire G between the rearward and forward sections. The top side of the feeder body 68 thus has a notch or recess extending transversely (Y) across its width and longitudinally (X) along the middle section 75. The recess and exposed middle section 75 of the feed channel 74 are sufficiently long to accommodate the tip of a user's thumb and sufficiently long to permit the user's thumb to move longitudinally (X) some amount (e.g., 0.5 to 5.0 cm) to advance the guidewire via friction between the thumb and guidewire G. The recess and middle section 75 should be sufficiently short to prevent buckling of the guidewire during manual feeding. The recess is also positioned so that the user's thumb can contact and move the guidewire when the user is holding the handle 22 via the grip 42. As the typical user will wear medical gloves while using the device, the friction inherently provided by the gloved thumb is put to good use in advancing the guidewire without the need for a dedicated actuator. It is also contemplated that the guidewire feed channel 74 could open on a different surface of the device housing 14 such that a different finger could advance the guidewire directly.

The above-described device 10 is useful in various methods as a part of medical procedures. For example, the device 10 may be useful in a method that includes placement of a guidewire in a blood vessel using only one hand. In another example, the device may be useful in a method that includes feeding a guidewire into a fluid-filled part of the body of a patient through a needle while the needle is coupled to a syringe. In another example, the device may be useful in a method that includes guiding the tip of a needle into a blood vessel, and then feeding a guidewire into the blood vessel through the needle, wherein real-time imagery is continuously provided before the needle is guided into the blood vessel, while the needle is guided into the blood vessel, and while the guidewire is being fed into the blood vessel through the needle, without interruption of the imagery.

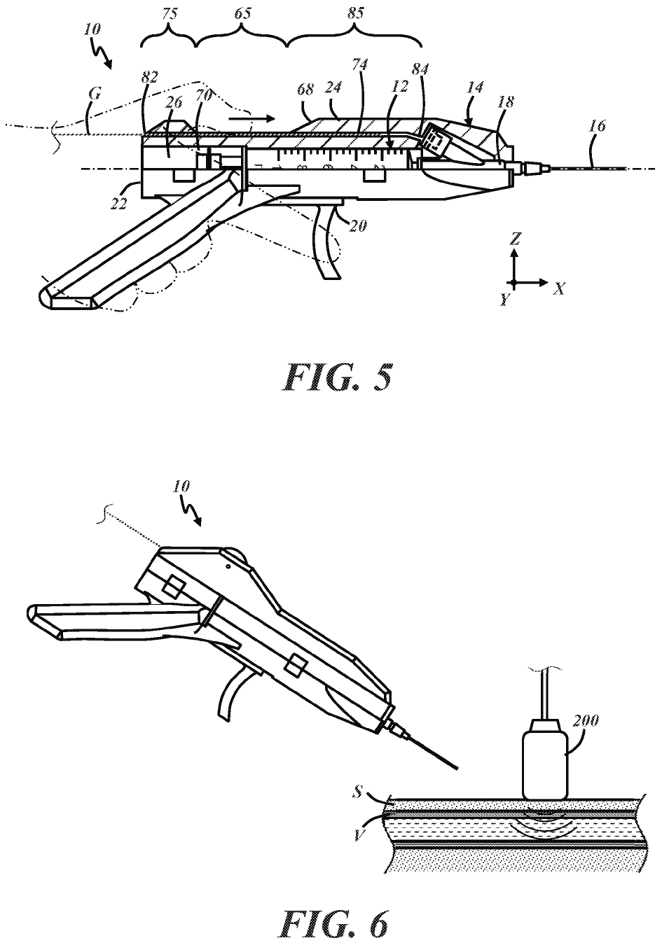

While not necessarily drawn to scale, FIGS. 6-11 schematically depict use of an embodiment of the above-described device 10 in an illustrative method based on the Seldinger technique. FIG. 6 depicts the device 10 preparing to pierce the skin(S) of a person to obtain vascular access through the wall of a blood vessel (V). An ultrasound transducer 200 is placed in contact with the skin over the intended access site to provide real-time imagery at the access site. The device 10 is held and maneuvered by one hand of the user, such as the dominant hand, while the transducer is held and maneuvered by the other hand of the user.

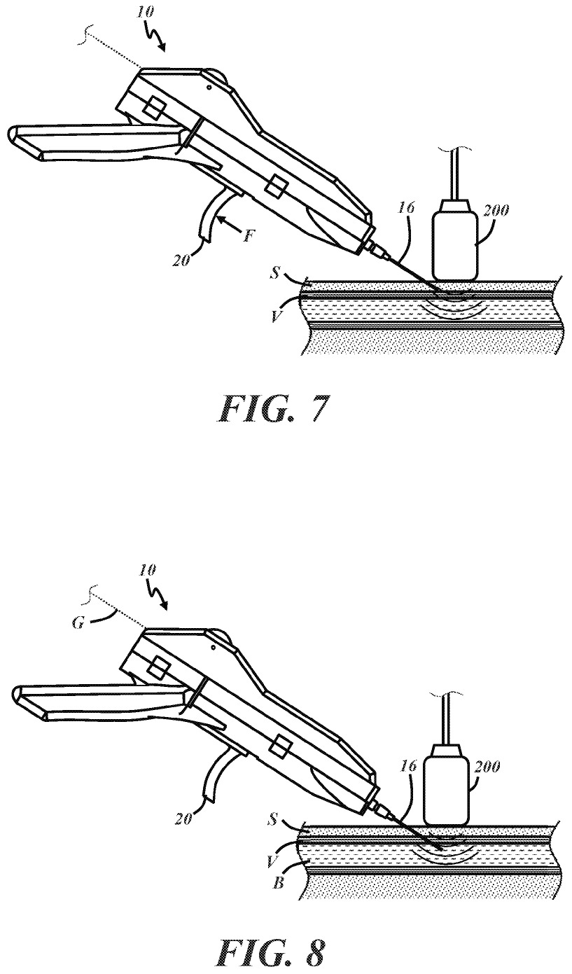

FIG. 7 depicts the device 10 after the needle 16 has pierced the skin(S) but has not yet pierced the blood vessel (V). While the needle is proceeding through the skin, the user of the device 10 can continually manipulate the transducer 200 to provide imagery showing where the tip of the needle is in relation to the blood vessel. At this stage, the user applies a force F to the actuator 20 to reduce the pressure within the syringe of the device in preparation for aspiration. In a conventional US-guided Seldinger technique, the person performing the procedure must hold and manipulate the syringe and needle, which involves using their dominant hand in a manner that permits the thumb of the dominant hand to pull back on the plunger of the syringe to create the negative pressure in the syringe. This leads to unnatural and awkward finger positions with some fingers on the barrel of the syringe and one or more fingers rearward of the flange of the barrel to prevent the needle from being extracted while the negative pressure in the syringe is achieved. The trigger actuator 20 of the illustrated device 10 permits more controlled and steady force to be applied to the plunger by the user and provides better tactile feel when aspiration occurs.

FIG. 8 depicts the device 10 after the tip of the needle 16 has pierced and been guided through the wall of the blood vessel (V) to achieve aspiration. The user of the device detects aspiration in multiple ways, including the loss of vacuum in the syringe leading to less force being required on the actuator to move the plunger of the syringe, visual detection of blood (B) in the syringe or connector 18, and confirmation that the tip of the needle 16 is in the blood vessel via real-time ultrasound imagery. At this stage, the user of the device releases the force F previously applied to the actuator in preparation for feeding the guidewire G into the blood vessel through the needle 16.

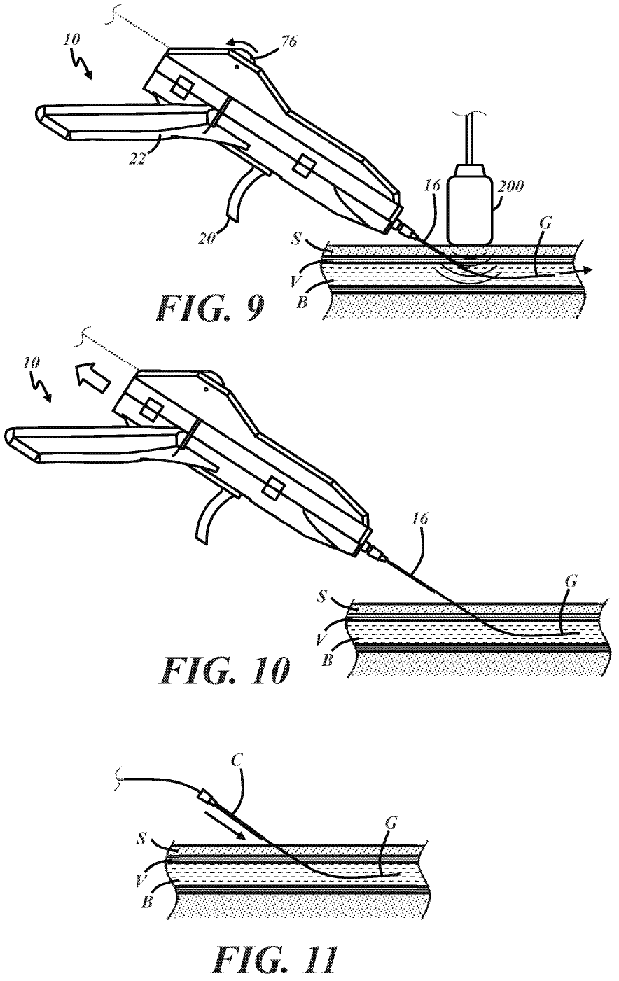

FIG. 9 depicts the device 10 after the guidewire G has been fed through the device 10 and into the vessel (V) through the tip of the needle 16. To achieve this, the user rotates the thumbwheel actuator 76 as depicted in FIG. 9 using the thumb of the same hand that is still holding the device 10 by the handle 22. This enables the user to continue to monitor the access site with real-time ultrasound imagery using the hand not holding the device 10 as the guidewire G is fed into the vessel (V) to confirm proper placement inside the vessel. Real-time imagery of the access site is thus available continuously and without interruption from the time the needle 16 pierces the skin(S) until the guidewire G is in the vessel (V). Alternatively, the user advances the guidewire directly using a thumb or other digit as discussed above in conjunction with FIG. 5.

In a conventional US-guided Seldinger technique, the person performing the procedure must hold the needle in place in the blood vessel with one hand while decoupling the syringe from the needle with the other hand. This requires the medical professional to release the ultrasound transducer and thereby lose the real-time imaging. A very high skill level and very fine motor skills are required to detach the syringe from the needle while hopefully maintaining the location of the tip of the needle in the blood vessel. Once the syringe is detached from the needle, some blood (B) from the vessel (V) escapes through the back end of the needle while the medical professional obtains the guidewire to prepare for insertion through the back end of the needle. Here again, fine motor skills are at work as the medical professional holds the needle in place in the blood vessel with one hand while feeding the guidewire through the needle with the other hand. Moreover, there is no confirmation that the guidewire has been properly placed in the blood vessel even after it is fed through the needle, as the tip of the needle could have been removed from the vessel while still under the skin without any indication.

The above-described medical device 10 can address all of these problems and more. Because guidance of the needle 16 into the blood vessel (V), aspiration, and advancement of the guidewire G are all performed with one hand, and with that one hand in essentially the same position with respect to the syringe during the entire time, the user's other hand is free to maneuver the ultrasound transducer 200 or other real-time image generator during the entire procedure. The device is also capable of improved guidewire placement even without ultrasonic guidance. Once the blood vessel is aspirated, the user of the device 10 can immediately begin feeding the guidewire into the vessel with a higher level of confidence that the needle has maintained its position in the blood vessel since there is no delay in which the syringe and needle are decoupled and in which the guidewire must be inserted manually into the back end of the needle. The procedure is also cleaner and safer with the device 10 since no blood escapes from the back end of the needle prior to guidewire advancement.

After the guidewire is properly placed, the needle 16 is withdrawn from the vessel (V) and skin(S), as in FIG. 10, and the entire length of the guidewire G is pulled from the forward end of the device 10 so that a catheter (or other device can be placed in the blood vessel by sliding it over the guidewire and into the access site created by the needle, as in FIG. 11. After catheter placement, the guidewire G is withdrawn from the vessel (V) through the catheter C.

While depicted obtaining vascular access in FIGS. 6-11, the device is useful for accessing any fluid-filled part of a patient's body that has no natural opening along the exterior of the body. In such cases, a tissue wall of the cavity must be pierced and the cavity aspirated to indicate successful access by the needle. In one example, the device 10 is used to access the pleural cavity surrounding the lungs for drainage and/or placement of a chest tube along the guidewire.

The primary contemplated use for the device 10 is central venous catheter placement, but vascular access, either veinous or arterial, is a common first step to many invasive procedures. A non-limiting list of procedures in which the above-described device 10 is useful includes placement of a triple lumen central venous catheter, a Cordis central venous catheter, a transvenous pacemaker, a Swan-Ganz catheter, or an arterial catheter. The device 10 is also useful for cardiac catheterization and percutaneous coronary intervention (PCI), interventional radiology (IR) embolization, IVC filter placement, thrombectomy, or placement of a percutaneous chest tube.

It is to be understood that the foregoing description is of one or more embodiments of the invention. The invention is not limited to the particular embodiment(s) disclosed herein, but rather is defined solely by the claims below. Furthermore, the statements contained in the foregoing description relate to the disclosed embodiment(s) and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art.

As used in this specification and claims, the terms “e.g.,” “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Further, the term “electrically connected” and the variations thereof is intended to encompass both wireless electrical connections and electrical connections made via one or more wires, cables, or conductors (wired connections). Other terms are to be construed using their broadest reasonable meaning unless they are used in a context that requires a different interpretation.

1. A medical device comprising a needle coupled with a syringe, the device being configured to feed a guidewire through the needle and out of a tip of the needle with only one hand and without decoupling the needle from the syringe.

2. The medical device of claim 1, further comprising:
a handle supporting the syringe; and
a guidewire feeder supported by the handle, the guidewire feeder comprising a body, a guide channel extending through the body, and a wheel,
wherein the wheel is operable by a hand of a user to feed the guidewire along the guide channel and through the needle while the same hand is holding the device by the handle.

3. The medical device of claim 1, further comprising:
a handle supporting the syringe; and
a guidewire feeder supported by the handle, the guidewire feeder comprising a body and a guide channel extending through the body,
wherein the guide channel opens on an exterior surface of the body such that a hand of a user can contact and move the guidewire in the channel to feed the guidewire along the guide channel and through the needle while the same hand is holding the device by the handle.

4. The medical device of claim 1, further comprising:
a handle supporting the syringe; and
an actuator supported by the handle and operable by a hand of a user to decrease pressure in the syringe when the tip of the needle is blocked and while the same hand is holding the device by the handle.

5. The medical device of claim 1, further comprising a connector and a check valve, the connector having first and second branches coupled with the needle, wherein the first branch is coupled with the syringe and the guidewire is fed to the needle via the second branch, and wherein the check valve is located along the second branch to prevent external fluid flow into the connector via the second branch when pressure is decreased in the connector.

6. The medical device of claim 1, further comprising:
a handle supporting the syringe;
a guidewire feeder operable to feed the guidewire through the needle;
an actuator operable to decrease pressure in the syringe when the tip of the needle is blocked;
a connector having first, second, and third ends interconnected by an internal volume of the connector; and
a check valve,
wherein the first end of the connector is coupled with the syringe, the second end of the connector receives the guidewire, the third end of the connector is coupled with the needle, and the check valve prevents external fluid flow into the internal volume through the second end of the connector when pressure is decreased in the internal volume of the connector.

7. The medical device of claim 6, wherein the feeder comprises a wheel operable by a hand of a user to feed the guidewire along a guide channel of the device, into the second end of the connector, through the check valve, out of the third end of the connector, and through the needle while the same hand is holding the device by the handle.

8. The medical device of claim 6, wherein the actuator comprises a trigger operable by a hand of a user to apply force to a plunger of the syringe to aspirate a fluid-filled part of the body when the tip of the needle accesses the fluid-filled part of the body through a wall of the fluid-filled part of the body while the same hand is holding the device by the handle.

9. The medical device of claim 1, further comprising a pressure transducer operable to detect pressure changes in the syringe during use of the device.

10. A method, comprising:
(a) guiding a tip of a needle through a wall of a fluid-filled part of a body of a person;
(b) maintaining a partial vacuum in the needle before and during step (a);
(c) feeding a guidewire into the fluid-filled part through the needle; and
(d) maintaining a position of the tip of the needle in the fluid-filled part during step (c),
wherein each of steps (a) through (d) are performed using only one hand.

11. The method of claim 10, further comprising use of real-time imagery during step (a), wherein the imagery is obtained at a location controlled by a different hand.

12. The method of claim 10, further comprising use of real-time imagery during steps (a) and (c), wherein the imagery is obtained at a location controlled by a different hand and the imagery is continuous between steps (a) and (c).

13. The method of claim 10, wherein the needle is coupled with a syringe during step (a), and step (c) is performed without decoupling the needle from the syringe.

14. The method of claim 10, wherein step (b) is performed using a digit of the one hand, and step (c) is performed using a different digit of the one hand.

15. The method of claim 10, wherein the fluid-filled part of the body is a blood vessel.

16. The method of claim 10, wherein the fluid-filled part of the body is a pleural cavity.

17. A method of obtaining vascular access, comprising:
(a) guiding a tip of a needle into a blood vessel of a person; and
(b) feeding a guidewire into the fluid-filled part through the needle,
wherein real-time imagery is continuously provided to a person performing the method during and between steps (a) and (b).

18. The method of claim 17, wherein steps (a) and (b) are performed using only one hand and the imagery is provided by an ultrasound transducer held in a different hand.

19. The method of claim 17, further comprising:
maintaining a partial vacuum in the needle before and during step (a); and
maintaining a position of the tip of the needle in the blood vessel during step (b),
wherein steps (a) and (b) and each maintaining step are performed using only one hand.

20. The method of claim 17, wherein steps (a) and (b) are performed while the needle is coupled with a syringe.
